SEQUENCE LISTING (3 sequences)

SEQ ID NO: 1 - DNA, 898 bp, Artificial Sequence
  AWE promoter containing a 387 bp fragment of the WAS alternative promoter immediately “upstream” of the 500 bp WAS proximal promoter present in the WE vector.
  misc_feature (1)..(898)
taagtcaaaggaggagagggcaacgcggtgggcaggagagaggccaacggccgcccggggcgaggggagccggtaggacgggaccaggactggccgacccggccccgcgcggggaagggggcgccttcctcccacaacacaaaacggtgcgcccgggttggccgcccctccccagtggtgcggccccgggtggacgcttccgtgcgcgcgtccatgcccagccattgcgggctgcgggctccaagggtcgcacacgctggagagtgcaggttgccgggtccacccacagggctgtagacacccctagggtcacacagacaaggctctggacacccacaggggcacacacattggggagtgggcactcctgggctcacaaagactgagaatcactagtgaattcgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggaggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaag

SEQ ID NO: 2 - DNA, 500 bp, Artificial Sequence
  500 bp fragment of the proximal WAS promoter
  misc_feature (1)..(500)
aattcgggattacaggtgtgagctattgtccccagccaaaaggaaaagttttactgtagtaacccttccggactagggacctcgggcctcagcctcaggctacctaggtgctttagaaaggaggccacccaggcccatgactactccttgccacagggagccctgcacacagatgtgctaagctctcgctgccagccagagggaggaggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaag

SEQ ID NO: 3 - DNA, 398 bp, Artificial Sequence
  387 bp fragment of the WAS alternative promoter
  misc_feature (1)..(398)
taagtcaaaggaggagagggcaacgcggtgggcaggagagaggccaacggccgcccggggcgaggggagccggtaggacgggaccaggactggccgacccggccccgcgcggggaagggggcgccttcctcccacaacacaaaacggtgcgcccgggttggccgcccctccccagtggtgcggccccgggtggacgcttccgtgcgcgcgtccatgcccagccattgcgggctgcgggctccaagggtcgcacacgctggagagtgcaggttgccgggtccacccacagggctgtagacacccctagggtcacacagacaaggctctggacacccacaggggcacacacattggggagtgggcactcctgggctcacaaagactgagaatcactagtg

(19) United States
(12) Patent Application Publication
(10) Pub. No.: US 2022/0009993 A1
(43) Pub. Date: Jan. 13, 2022

(54) POLYNUCLEOTIDE FOR SAFER AND MORE EFFECTIVE IMMUNOTHERAPIES

(71) Applicant: FUNDACIÓN PÚBLICA ANDALUZA PROGRESO Y SALUD, Sevilla (ES)

(72) Inventors: Francisco MARTIN MOLINA, Sevilla (ES); María TRISTÁN MANZANO, Sevilla (ES); Noella MALDONADO PÉREZ, Sevilla (ES); Karim BENABDELLAH EL KHLANJI, Sevilla (ES); Pilar MUNOZ FERNÁNDEZ, Sevilla (ES)

(21) Appl. No.: 17/293,845

(22) PCT Filed: Nov. 14, 2019
(86) PCT No.: PCT/EP2019/081346

§ 371 Date: May 13, 2021

(30) Foreign Application Priority Data
Nov. 14, 2018 (EP) 18380016.8

(51) Int. Cl. C07K 14/725; C12N 15/86; C12N 5/0783

(52) U.S. Cl. CPC C07K 14/7051; C12N 15/86; C12N 2830/008; C12N 2501/515; C12N 2510/00; C12N 5/0636

(57) ABSTRACT

The present invention provides polynucleotides and viral vectors for transfection of a mammalian host cell, preferably lentiviral vectors, encoding at least one CAR (chimeric antigen receptors)and a promoter from the Wiskott-Aldrich syndrome locus, in particular the promoter of SEQ. ID NO 1, operably linked to the CAR in order to drive its expression.

### FIELD OF THE INVENTION

The present invention relates to a new technology to generate immunotherapeutic T cells. In particular, the invention provides an improved system to generate immunotherapeutic T cells comprising a chimeric antigen receptor (CAR).

### BACKGROUND OF THE INVENTION

Adoptive immunotherapy, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering (June, C. and Sadelain, M. Chimeric Antigen Receptor Therapy NEJM 2018:379(1);64-73). Transfer of viral antigen specific T cells is a well-established procedure used for the treatment of transplant associated viral infections and rare viral-related malignancies. Similarly, isolation and transfer of tumor specific T cells has been shown to be successful in treating melanoma.

Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signalling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signalling domains for first generation CARs are derived from the cytoplasmic region of the CD3zeta or the Fc receptor gamma chains. First generation CARs have been shown to successfully redirect T-cell cytotoxicity, however, they failed to provide prolonged expansion and antitumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010). CD19 has been presented as an attractive target for immunotherapy because the vast majority of B-acute lymphoblastic leukemia (B-ALL) uniformly express CD19, whereas expression is absent on non-hematopoietic cells, as well as myeloid, erythroid, T cells and bone marrow stem cells. Clinical trials targeting CD19 on B-cell malignancies are underway with encouraging anti-tumor responses. Most infuse T cells genetically modified to express a chimeric antigen receptor (CAR) with specificity derived from the scFv region of a CD19-specific mouse monoclonal antibody FMC63 (Nicholson, Lenton et al. 1997; Cooper, Topp et al. 2003; Cooper, Jena et al. 2012) (International application: WO2013/126712). However, there is still a need to improve construction of CARs that show better compatibility with T-cell proliferation, in order to allow the cells expressing such CARs to reach a significant clinical advantage. In this sense and in spite of the clear-cut benefit for the patients treated with CAR-T, actual technologies using strong promoters to express CARs comes with a down side. Severe side effects, including patient deaths, have been reported mainly due to a cytokine release syndrome (CRS) associated with hyper-activity of the CAR-T cells in the first days after infusion. In addition, a significant percentage of patients that responded initially, relapsed as a consequence of reduce longevity (and efficacy) of administrated CAR-T cells. Eyquem, Mansilla-Soto et al. have already demonstrated that TCR-like expression improve anti-leukemic activity of CAR-T cells using genome editing systems to express transgenes through the TRAC locus promoter. However, genome editing strategies use very sophisticated technologies difficult to implement in clinical practice.

We have thus tested a panel of different LV (lentiviral vectors) backbones to investigate the transgene expression profiles on T cells at different times after TCR activation and compare such expression pattern with the TCR expression profile. Our data showed that the AW backbone, expressing the transgene through a chimeric promoter from the Wiskott-Aldrich syndrome locus follows closely the expression of the TCR. Importantly, contrary to the other promoters analysed (EF1a, CMV,) the AW fails to increase the expression levels of the transgene upon TCR activation, which is of especial relevance to reduce CRS intensity.

### BRIEF DESCRIPTION OF THE FIGURES

FIG. 1. Scheme of the AW LVs backbone (Ref Frecha et al. Gene Ther. 2008 Jun;15(12):930-41). The Chimeric WAS promoter is represented by purple arrows. The transgene (i.e. CAR) is represented by a red arrow. LTR from HIV-1 are represented by the dashed terminal arrows.

FIG. 2. CD3 surface expression in human T cells after CD3/CD28 stimulation. A) Scheme showing the stimulation of T cells using TransAct Reagent (Miltenyi), a nanomatrix of anti-CD3/CD28 molecules that would mimic TCR intracelullar signaling in a physiological situation. B) Drawing showing the experiment procedure: CD3 expression was measured by flow cytometry at 0, 8, 24, 48, 96 h and 7 days after stimulation using anti-CD3-PerCP-Cy5 (OKT3 clon, Biosciences 1:100). C) Graph showing CD3 expression levels analyzed at the different time points. Data represent the ratio of the Median of Fluorescence Intensity (MeFI) of the CD3+ population related to the CD3− population at each time point.

FIG. 3. Schematic representation of the different lentiviral vectors in the study. All the LVs are constructed in a self-inactivated (SIN) backbone expressing the enhanced Green Fluorescence Protein (eGFP) under diferent promoters: AWE LVs drive eGFP expression through the chimeric endogenous promoter from the Wiskott-Aldrich Syndrome gene; EFEWP LV, under the control of the elongation factor 1-alpha (EF1α) promoter and CEWP LV under the citomegalovirus CMV) promoter. The WPRE has been removed from the AWE LV backbone to reduce expression levels and to better control expression levels.

FIG. 4. The AWE LV mimics the expression profile of the TCR. A) Scheme showing the experiment set up. T cells were stimulated with TransAct reagent during 48h and transduced with the different LVs at day −10. 10 days later the T cells were analyzed for TCR (CD3-PerCP-Cy5 (OKT3, eBiosciences 1:100) and eGFP expression by FACS (Day 0). The cells were then stimulated again with TransAct and analyzed at 8 h, 24 h, 48 h, 72 h and 96 h. B) Graph showing CD3 (Black circles) and eGFP (color symbols) expression levels analyzed at the different time points. Data represent the ratio of the Median of Fluorescence Intensity (MeFI) of the positive population related to the negative population in the density plots at each time point. C) Graph showing the kinetic of CD3 (Black circles) and eGFP (color symbols) expression at different time points related to 0 h. The same data as in B) is represented to compare the upregulation or downregulation of the expression levels in T cells at the different time points after TCR stimulation. The AWE LVs is the only LV that lower the expression of the transgene upon TCR activation. Both, the EFWP and the CEWP LV increased the expression 3-4 times 24-48 h post TCR activation. Most T-CARs express the CAR through the EF1alfa promoter. Our hypothesis is that the AWE LV backbone is a good alternative to express CARs due to this TCR-like expression pattern.

FIG. 5. Expression kinetic of the TCR(CD3) compared to the different LVs. T cells were stimulated with TransAct reagent during 48 h and transduced with the different LVs at day −10. 10 days later, the T cells were analyzed for TCR (CD3-PerCP-Cy5 (OKT3, eBiosciences 1:100)) and eGFP expression by FACS (Day 0). The cells were then stimulated again with TransAct and analyzed at 8 h, 24 h, 48 h and 72 h. The graph show the changes in CD3 (Black circles) and eGFP (color symbols) expression related to day 0 at 8 h, 24 h, 48 h and 72 h. (Two-tailed T student, p<0.05,*; p<0.01,**; p<0.001,***. At least three indepent experiments were performed).

FIG. 6. Physiological stimulation of T cells generates a downregulation of T cell receptor (TCR). Isolated primary T cells (CD3+) were stimulated with a nanomatrix of anti-CD3/CD28 and CD3 expression on the surface (b), FACs analysis) and mRNA levels (c) were determined at indicated time points (a).

FIG. 7. CAR expression driven by the EF1-α-promoter is increased after stimulation via TCR and CD19 pathways. a) Scheme indicating the three possible activation pathways: anti-CD3/CD28 (that target only TCR, right); MHC-TCR binding of an antigen presenting cells (e.g

B cell, macrophage . . . center); and CAR signaling after the interaction of CD19 (B cells)-Anti-CD19 CAR (T cell, left). b) Representative FACs histograms of the CAR expression driven by EF1-α-promoter-CAR transduced T cells that shown an increment of both percentage of CAR positive cells and CAR expression after stimulated through the three different methods described above (a). HL-60, a promonocytic cell line, was used as CD19− (negative) cells.

FIG. 8. CAR expression kinetics after stimulation. a) CAR-Lentiviral backbones used in that project. ARI, a second-generation CAR that is express under the control of EF1-alpha, was kindly provided by Dr. Manel Juan (developed and patented by the Hospital Clinic Barcelona). WARI uses de SEQ ID NO 2 (W0.5 promoter) to express the CAR and AW uses SEQ ID NO 1 (AW promoter). b) Primary T cells were activated during 48 h with anti-CD3/CD28 nanomatrix prior LV-transduction. Cells were let them rest for 10 days before stimulating with HL-60 cells (TCR pathway) or Nalm6 cells (CD19+, CAR/TCR pathway). c) CAR was stained with anti-murine Fab-biotin and streptavidin-PE at indicated times after activation and % CAR positive cells are represented related to 0 h. ARI showed a significant increase whereas AWARI mimicked better the CD3 profile (black lines). Unpaired T-Test, two tails. p<0.01, **.

FIG. 9. AWARI CAR-T cells lysed CD19+ in vitro and in vivo. a) In vitro lysis experiment. Briefly, CD19+ cells Nalm6 and Namalwa were co-cocultured with NT (no-transduced cells), ARI and AWARI-T cells in V-bottom plates and specific lysis was determined after 48 h, comparing the % of live target cells given by ARI/AWARI with that percentage given by NT cells (non-CAR specific lysis). b) AWARI-T cells were able to lysed CD19+ cells (˜70-80% lysis) in vitro. c) Exhaustion was determined by the surface expression of Tim3. AWARI T cells exhibited less Tim3+ cells after 48 h of co-culture with CD19+ cells. d) 3×105 Namalwa cells that express eGFP and Nluciferase were inoculated intravenously (IV) in NSG3GM-mice. 3 days later, 5×106 cells of NT, ARI, AWARI T cells (expressing a 30% and 25% of CAR+ cells, respectively) were inoculated IV and bioluminescence analysis (BLI) were performed up to 10 days. e) BLI images at day 10 after T cells infusion (control, non-treated mice, only Namalwa; NTD, Non-transduced T cells+ Namalwa) were acquired on an IVIS Spectrum In Vivo Imaging System, PerkinElmer after administration intraperitoneally of the Nanoluc substrate (Promega). f) Photon quantification of BLI for every mice group. Paired T test, two tails. p<0.05, *. P<0.01, **. g) Mice were sacrificed at day 15, and the presence of Namalwa cells (humanCD19+eGFP+ cells) were determined by FACS in bone marrow, spleen and liver, showing that both ARI and AWARI efficiently lysed CD19+ cells in vivo.

### DESCRIPTION OF THE INVENTION

Unless specifically defined herein, all technical and scientific terms used have the same meaning as commonly understood by a skilled artisan in the fields of gene therapy, biochemistry, genetics, and molecular biology. All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrooket al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods I n ENZYMOLOGY (J. Abelson and M. Simon, eds. -in-chief, Academic Press, Inc., New York), specifically, Vols.154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The inventors have generated specific lentiviral vectors (LV), preferably having a self-inactivated (SIN) backbone, expressing a chimeric antigen receptor (CAR) under different promoters. In particular, we have tested LVs that drive CAR expression made by using the AWE promoter containing a 387 bp fragment of the WAS alternative promoter immediately “upstream” of the 500 bp WAS proximal promoter present in the WE vector (SEQ ID NO 1); the EFEWP lentiviral vector under the control of the elongation factor 1-alpha (EF1α) promoter and the CEWP lentiviral vector under the citomegalovirus (CMV) promoter, and we have surprisingly found that introduction of the resulting CARs into primary T cells indicates that only the lentiviral vector containing the AWE promoter of SEQ ID NO 1, follows closely the expression of the TCR. Importantly, and contrary to the other promoters analyzed (EF1a, CMV), the AWE did not increase the expression levels of the transgene upon TCR activation, which is certainly of especial relevance to reduce CRS intensity.

The present invention thus provides polynucleotides and viral vectors for transfection of a mammalian host cell, preferably lentiviral vectors, encoding the above described CAR and a promoter from the Wiskott-Aldrich syndrome locus, in particular, the promoter of SEQ ID NO 1, operably linked to the CAR in order to drive its expression. In a preferred embodiment, the present invention relates to a polynucleotide or vector comprising a promoter that drives the expression of the CAR, having at least 70%, preferably at least 80%, more preferably at least 90%, 95% 97%, 99% or 100% sequence identity with a fragment of SEQ ID NO 1 that comprises nucleotide 388 to nucleotide 887 (SEQ ID NO 2) of said sequence. In another preferred embodiment, the present invention relates to a polynucleotide or vector comprising a promoter that drives the expression of the CAR, having at least 70%, preferably at least 80%, more preferably at least 90%, 95% 97%, or 99% sequence identity with SEQ ID NO 1.

In a preferred embodiment, said polynucleotides are included in lentiviral vectors in view of being stably expressed in the cells.

To direct, transmembrane polypeptide into the secretory pathway of a host cell, a secretory signal sequence (also known as a leader sequence, prepro sequence or pre sequence) can be provided in the polynucleotide sequence or vector sequence of the invention. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). Those skilled in the art will recognize that, in view of the degeneracy of the genetic code, considerable sequence variation is possible among these polynucleotide molecules. Preferably, the nucleic acid sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

Methods of Engineering an Immune Cell:

In an encompassed particular embodiment, the invention relates to a method of preparing immune cells for immunotherapycomprising the introduction into said immune cells the polynucleotide or vector according to the present invention and expanding said cells. In particular embodiment, the invention relates to a method of engineering an immune cell that comprises providing a cell and expressing at the surface of said cell at least one CAR as described above. In a particular embodiment, the method comprises transforming or transducing the cell with at least one polynucleotide or vector encoding CAR as described above, and expressing said polynucleotides into said cell.

In another embodiment, said method further comprises a step of genetically modifying said cell by inactivating at least one gene expressing one component of the TCR, a target for an immunosuppressive agent, HLA gene and/or an immune checkpoint gene such as PD1 or CTLA-4. In a preferred embodiment, said gene is selected from the group consisting of TCRalpha, TCRbeta, CD52, GR, PD1 and CTLA-4. In a preferred embodiment said method further comprises introducing into said T cells a rare-cutting endonuclease able to selectively inactivate by DNA cleavage said genes. In a more preferred embodiment said rare-cutting endonuclease is TALE-nuclease or Cas9 endonuclease.

Delivery Methods

The different methods described above involve introducing CAR into a cell by using expression vectors. As non-limiting example, said CAR can be introduced as transgenes encoded by one lentiviral vector.

Immune Cells

The present invention also relates to isolated cells or cell lines susceptible to be obtained by said method to engineer cells. In particular said isolated cell comprises at least one CAR and a promoter from the Wiskott-Aldrich syndrome locus, in particular of SEQ ID NO 1, operably linked to the CAR in order to drive its expression. In another embodiment, said isolated cell comprises a population of CARs and promoters from the Wiskott-Aldrich syndrome locus, in particular of SEQ ID NO 1, operably linked to the CARs in order to drive their expression, each one comprising different extracellular ligand binding domains. Immune cells of the present invention are activated and proliferate independently of antigen binding mechanisms.

In the scope of the present invention is also encompassed an isolated immune cell, preferably a T-cell obtained according to any one of the methods previously described. Said immune cell refers to a cell of hematopoietic origin functionally involved in the initiation and/or execution of innate and/or adaptative immune response. Said immune cell according to the present invention can be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. Said isolated cell can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. In another embodiment, said cell can be derived from the group consisting of CD4+ T-lymphocytes and CD8+ T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor, from a patient diagnosed with cancer or from a patient diagnosed with an infection. In another embodiment, said cell is part of a mixed population of cells which present different phenotypic characteristics. In the scope of the present invention is also encompassed a cell line obtained from a transformed T-cell according to the method previously described. Modified cells resistant to an immunosuppressive treatment and susceptible to be obtained by the previous method are encompassed in the scope of the present invention.

Activation and Expansion of T Cells

Whether prior to or after the generation of the transformed or transduced T cells, even if the modified immune cells of the present invention are activated and proliferate independently of antigen binding mechanisms, the immune cells, particularly T-cells of the present invention can be further activated and expanded generally using methods as described, for example, in U.S. Pat. Nos 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo. Generally, the T cells of the invention are expanded by contact with an agent that stimulates a CD3/TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemaglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, -2, 11-15, TGF, and TNF- or any other additives for the growth of cells. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics.

In another particular embodiment, said cells can be expanded by co-culturing with tissue or cells. Said cells can also be expanded in vivo, for example in the subject's blood after administrating said cell into the subject.

Therapeutic Applications

In another embodiment, isolated cells obtained by the different methods or cell line derived from said isolated cell as previously described can be used as a medicament. In another embodiment, said medicament can be used for treating cancer, particularly for the treatment of B-cell lymphomas and leukemia in a patient in need thereof. In another embodiment, said isolated cell according to the invention or cell line derived from said isolated cell can be used in the manufacture of a medicament for treatment of a cancer in a patient in need thereof.

In another aspect, the present invention relies on methods for treating patients in need thereof, said method comprising at least one of the following steps:
(a) providing an immune-cell obtainable by any one of the methods previously described;
(b) Administrating said transformed immune cells to said patient.

On one embodiment, said T cells of the invention can undergo robust in vivo T cell expansion and can persist for an extended amount of time.

Said treatment can be ameliorative, curative or prophylactic. It may be either part of an autologous immunotherapy or part of an allogenic immunotherapy treatment. By autologous, it is meant that cells, cell line or population of cells used for treating patients are originating from said patient. By allogeneic is meant that the cells or population of cells used for treating patients are not originated from said patient but from a donor.

Cells that can be used with the disclosed methods are described in the previous section. Said treatment can be used to treat patients diagnosed with cancer. Cancers that may be treated may comprise nonsolid tumors (such as hematological tumors, including but not limited to pre-B ALL (pedriatic indication), adult ALL, mantle cell lymphoma, diffuse large B-cell lymphoma and the like). Types of cancers to be treated with the CARs of the invention include, but are not limited to certain leukemia or lymphoid malignancies. Adult tumors/cancers and pediatric tumors/cancers are also included. It can be a treatment in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administered into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient. The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p7056 kinase that is important for growth factor induced signaling (rapamycin) (Henderson, Naya et al. 1991; Liu, Albers et al. 1992; Bierer, Hollander et al. 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery. Other definitions
Unless otherwise specified, “a,” “an,” “the,” and “at least one” are used interchangeably and mean one or more than one. Amino acid residues in a polypeptide sequence are designated herein according to the one-letter code, in which, for example, Q. means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution. Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.

“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogues of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
By chimeric antigen receptor (CAR) is intended molecules that combine a binding domain against a component present on the target cell, for example an antibody-based specificity for a desired antigen (e.g., tumor antigen) with a T cell receptor-activating intracellular domain to generate a chimeric protein that exhibits a specific anti-target cellular immune activity. Generally, CAR consists of an extracellular single chain antibody (scFv) fused to the intracellular signaling domain of the T cell antigen receptor complex zeta chain (scFv) and have the ability, when expressed in T cells, to redirect antigen recognition based on the monoclonal antibody specificity.
By “ delivery vector” or “ delivery vectors” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides. In these cases, delivery vectors are molecule carriers. By “delivery vector” or “delivery vectors” is also intended delivery methods to perform transfection.
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semisynthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adenoassociated viruses), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox a ndcanarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as non-limiting example, that are able to integrate the genome of a target cell. At the opposite by “non-integrative lentiviral vectors (or NILV)” is meant efficient gene delivery vectors that do not integrate the genome of a target cell through the action of the virus integrase.
Delivery vectors and vectors can be associated or combined with any cellular permeabilization techniques such as sonoporation or electroporation or derivatives of these techniques. By cell or cells is intended any eukaryotic living cells, primary cells and cell lines derived from these organisms for in vitro cultures.
By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines.

As non limiting examples cell lines can be selected from the group consisting of CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

All these cell lines can be modified by the method of the present invention to provide cell line models to produce, express, quantify, detect, study a gene or a protein of interest; these models can also be used to screen biologically active molecules of interest in research and production and various fields such as chemical, biofuels, therapeutics and agronomy as non-limiting examples.
by “mutation” is intended the substitution, deletion, insertion of up to one, two, three, four, five, six, seven, eight, nine, ten, eleven, twelve, thirteen, fourteen, fifteen, twenty, twenty five, thirty, fourty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “variant(s)”, it is intended a repeat variant, a variant, a DNA binding variant, a TALE-nuclease variant, a polypeptide variant obtained by mutation or replacement of at least one residue in the amino acid sequence of the parent molecule.
by “functional variant” is intended a catalytically active mutant of a protein or a protein domain; such mutant may have the same activity compared to its parent protein or protein domain or additional properties, or higher or lower activity. “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.
“similarity” describes the relationship between the amino acid sequences of two or more polypeptides. BLASTP may also be used to identify an amino acid sequence having at least 70%, 75%, 80%, 85%, 87.5%, 90%, 92.5%, 95%, 97.5%, 98%, 99% sequence similarity to a reference amino acid sequence using a similarity matrix such as BLOSUM45, BLOSUM62 or BLOSUM80. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means. A polynucleotide encoding such a functional variant would be produced by reverse translating its amino acid sequence using the genetic code.
“signal-transducing domain” or “co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD3OL, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, among others, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor.

A “co-stimulatory signal” as used herein refers to a signal, which in combination with primary signal, such as TCR/CD3 ligation, leads to T cell proliferation and/or upregulation or downregulation of key molecules. The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. Thus examples of cell surface markers that may act as ligands include those associated with viral, bacterial and parasitic infections, autoimmune disease and cancer cells.

The term “subject” or “patient” as used herein includes all members of the animal kingdom including non-human primates and humans.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

Examples

1.1. Construction of eGFP Expression Vectors

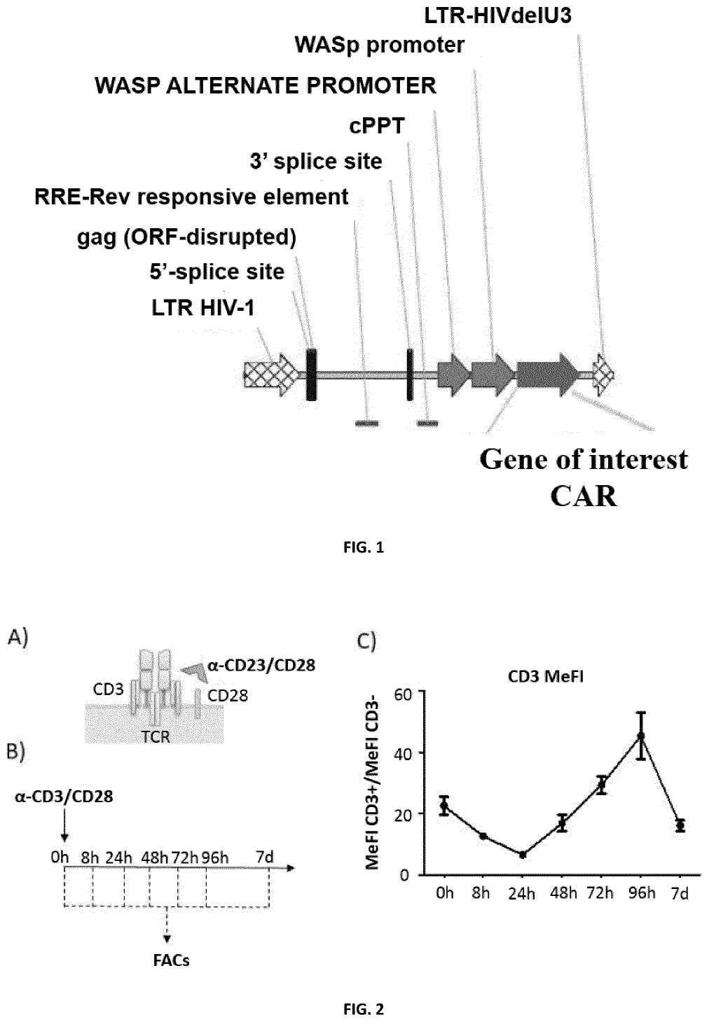

The expression of the AW gene, in T cells, is directed through two sequences with promoter activity (promoters). A sequence of approximately 1600 bp from the transcription initiation site, called the proximal promoter of the WAS gene and another one located 6 kb in the 5′ direction of the first one, called the alternative promoter of AW, FIG. 1A). In FIG. 1A the construction diagram of the lentiviral vectors used in the present invention is shown. As seen in said figure, the lentiviral vector WE contains a 500 bp fragment of the proximal WAS promoter that directs the expression of the selected transgene (the CAR protein), as described in: Martin, Toscano et al. to the. 2005; Toscano, Frecha et al. 2008; Toscano, Benabdellah et al. 2009. On the other hand, the lentiviral vector AWE contains a 387 bp fragment of the WAS alternative promoter immediately “upstream” of the 500 bp WAS proximal promoter present in the WE vector (SEQ ID NO 1), as described in: Martin, Toscano et al. 2005; Toscano, Frecha et al. 2008. All vectors share the autoinactivatable region “self-inactivated (SIN) lentiviral backbone” described by (Zufferey, Dull et al., 1998). In the vector pLVTHM, the GFP transgene is expressed under the constitutive EFI-ot promoter (htt:/′www.Addgene.Org/12247) and the CE vector expresses the GFP transgene under the control of the constitutive promoter of cytomegalovirus (CMV).

1.2. Production of Vectors and Transduction of T Cells

The lentiviral vectors were produced by the co-transfection of the 293T cells with three plasmids: (1) plasmid vector (WE, AWE, CE, and pLVTHM), (2) packaging plasmid (pCMVAR 8.91) and (3) plasmid enveloped VSV-G (pMD2.G), as described in Toscano, Frecha et al. 2004. The packaging and sheath plasmids used were obtained from http://www.addgene.org/Didier Trono. The day before transfection, 293T cells were plated in Petri dishes treated (Sarstedt, Newton, N.C.), to ensure exponential growth and 90% confluence. The plasmids pCMVAR 8.91 and pMD2.G were resuspended in 1 ml of DMEM (Biowest) together with 45 ul LipoD (Signagen) (proportions of plasmid 3:2:1). This mixture was added to the cell culture, previously washed with DMEM. Viral supernatants were collected, filtered through pores with a diameter of 0.45 μιη (Nalgene, Rochester, N.Y.), concentrated by ultracentrifugation (BeckmanCoulter) and resuspended in TexMACs (Milteny) culture medium.

For T cells transduction, cells were isolated by negative selection and activated using TransAct Reagent (Miltenyi), a nanomatrix of anti-CD3/CD28 molecules that would mimic TCR behavior in a physiological situation. 24 hours after stimulation, T cells were incubated with LV at MOI=10.

1.3 TCR Expression Profile Upon Activation

T cells were stimulated with TransAct reagent and analyzed at 8 h, 24 h, 48 h, 72 h and 96 h for CD3 surface expression using anti-CD3 monoclonal antibodies (CD3-PerCP-Cy5 (OKT3, eBiosciences 1:100) and FACs analysis.

1.4 Expression Profile of LVs Upon TCR Stimulation

T cells were stimulated with TransAct reagent during 48 h and transduced with the different LVs at day −10. 10 days later, the T cells were analyzed for TCR (CD3-PerCP-Cy5 (OKT3, eBiosciences 1:100) and eGFP expression by FACS (Day 0). The cells were then stimulated again with TransAct and analyzed at 8 h, 24 h, 48 h, 72 h and 96 h for both, eGFP and CD3 at each time point.

Results

TCR (CD3) Expression on T Cells Is Downregulated Upon Stimulation

T cell activation is a fine-tune process regulated by multiple mechanism that render different responses of the T cell. It is well known that the TCR at the surface is downregulated upon TCR engagement, controlling hyper-activation and/or exhaustion of the T cells. In order to see if we could mimic the process in the laboratory, we stimulated of T cells, isolated by negative selection, using TransAct Reagent (Miltenyi), and analyzed CD3 expression by flow cytometry at 0, 8, 24, 48, 96 h and 7 days after stimulation. Our data showed that, as expected, that the TCR levels were down-modulated at 8 h and 24 h post stimulation (FIG. 2C) and start to recover at 48 h, reaching a new peak of expression at 96 h.

The AWE LVs Mimic the Expression Profile of the TCR in T Cells

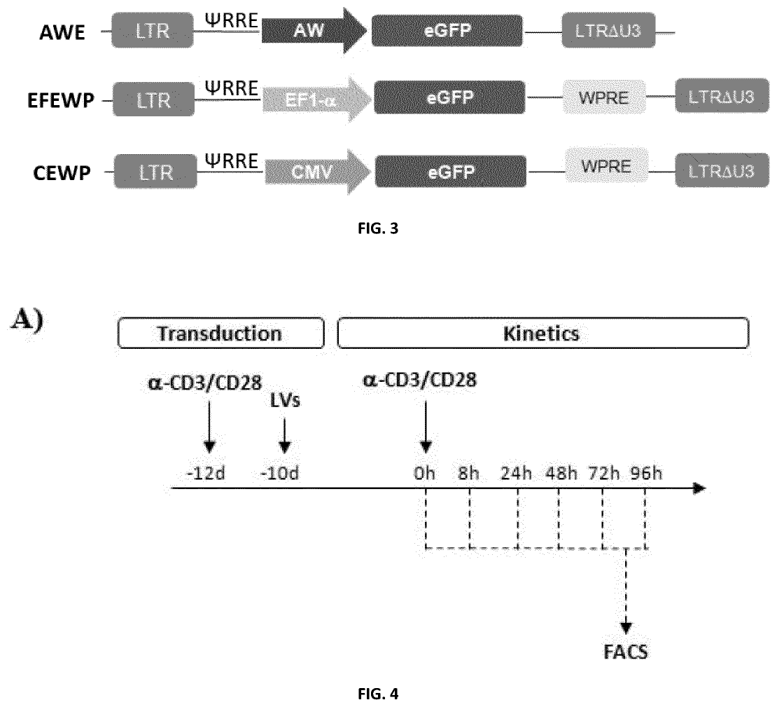

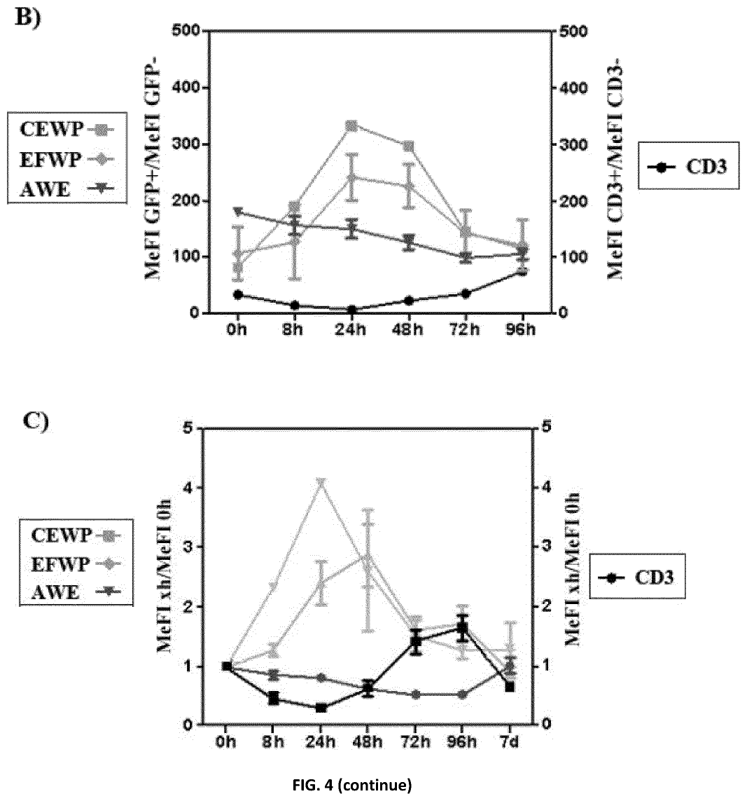

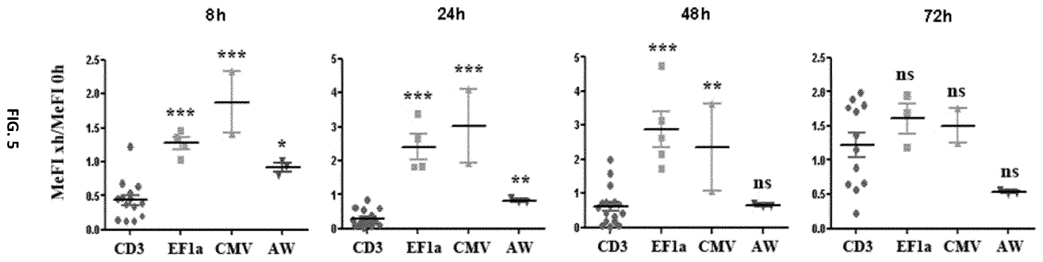

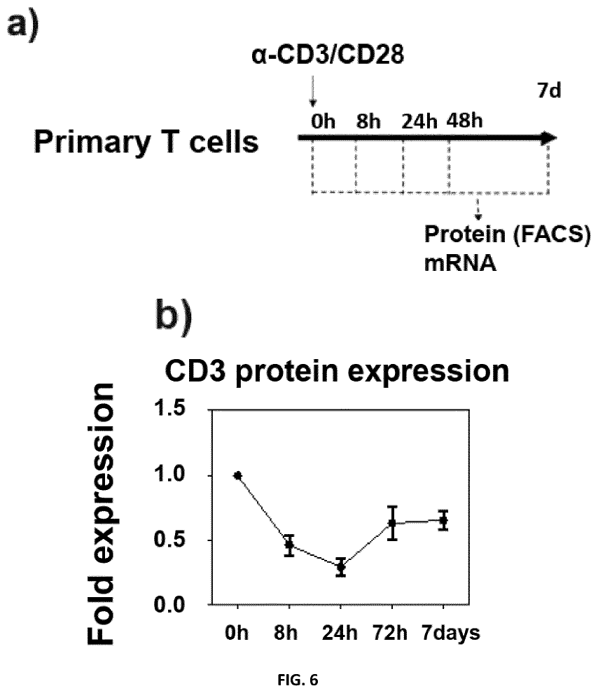

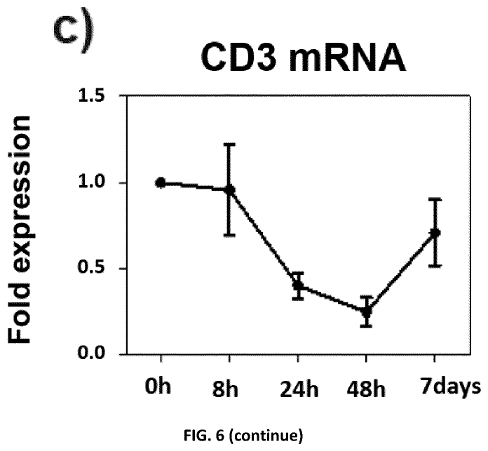

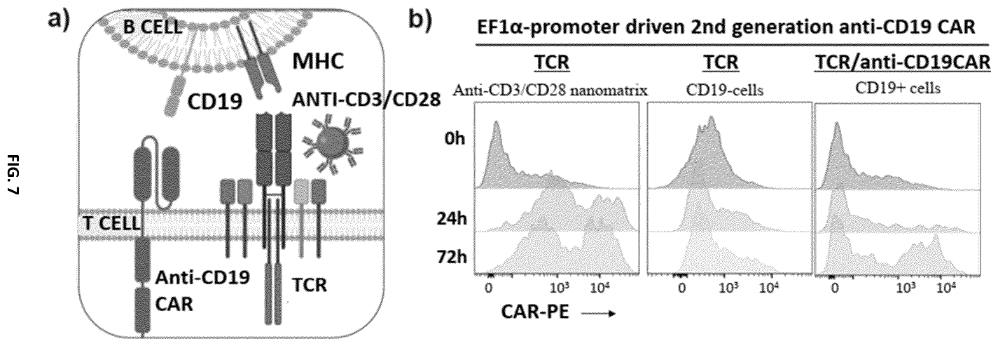

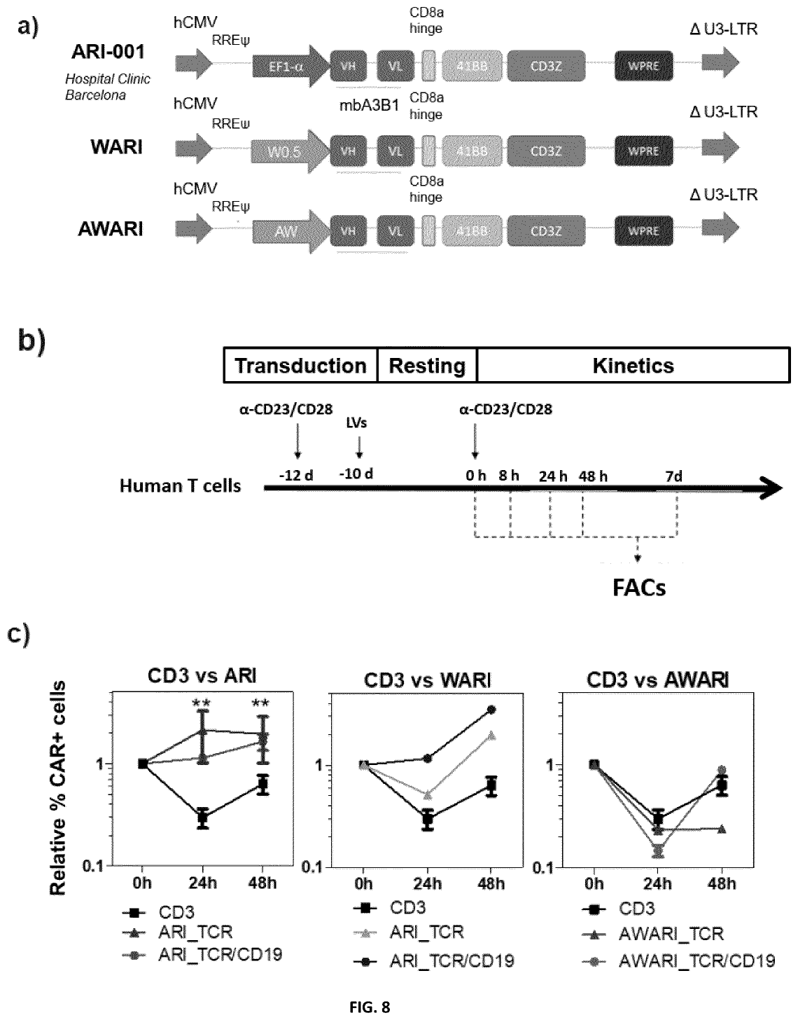

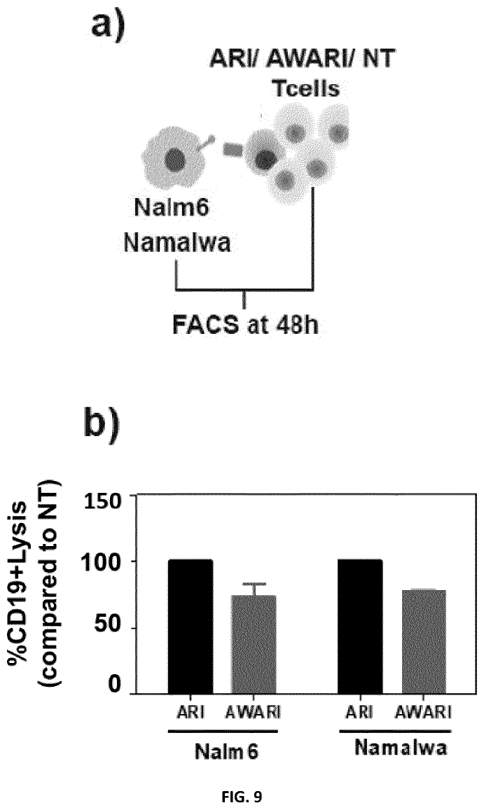

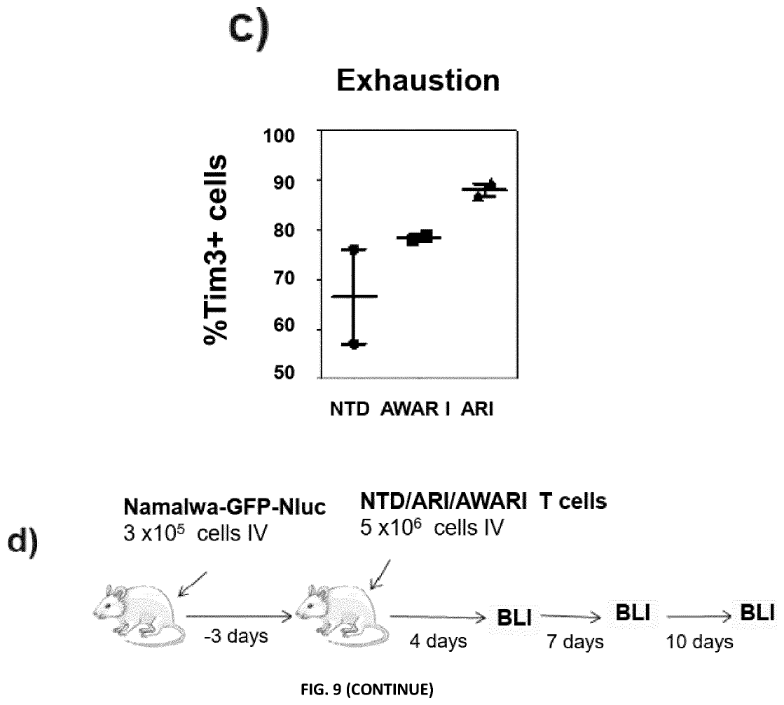

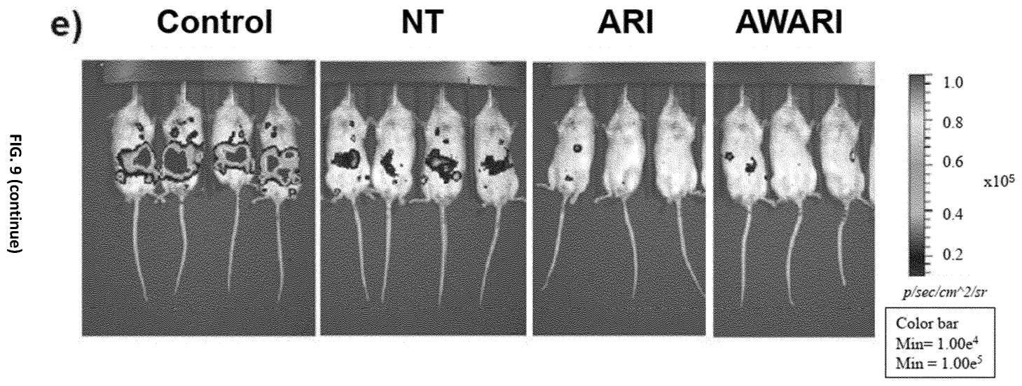

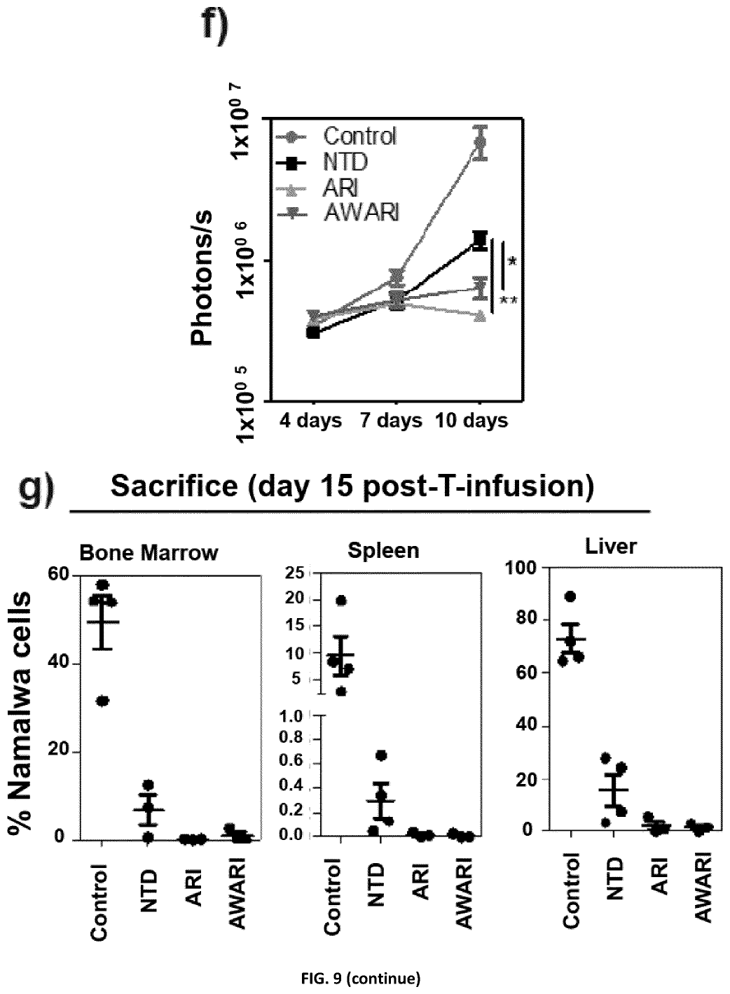

The group of Dr Sadelain demonstrated that the expression of a CAR following a TCR pattern improves the therapeutic efficacy of CAR-T cells (Eyquem, Mansilla-Soto et al. Targeting a CAR to the TRAC locus with CRISPR/Cas9 enhances tumour rejection. Nature 2017:543(7643);113-117). However, in their approach, in order to achieve TCR-like expression of the CAR gene, the author used genome editing strategies, which are very sophisticated technologies that are difficult to implement in the clinic. We hypothesized that we can use physiologically regulated LVs to mimic a TCR-like expression, a system that nowadays have a much easier clinical translation that genome edition. We therefore analyzed a panel of LVs expressing eGFP through different promoters (FIG. 3). We used the different LVs to transduce T cells and, as indicated in FIG. 4A, the cells were analyzed at different time points for eGFP and CD3 expression. We focus our attention in changes on eGFP in the first 96 hours post-stimulation, since this is the time in which we observed changes on TCR expression. As can be observed in FIGS. 4B and 4C only the AWE LV (pink line) follows the downregulation observed in the TCR (black line) 24 hours post-stimulation. The other LVs, including the EFWP LVs (widely used in CAR T cell therapies) increased their expression levels at this time point. FIG. 5 shows a statistical analysis of changes in expression at the different time points related to time=0. Again the AWE is the LV that more closely mimic the changes in TCR expression observed in T cells. These data showed that the AWE LV could also be used to achieve a TCR-like expression pattern of any transgen.

Based on this data, we propose the AWE LV as a new tool to express CARs on T cells for immunotherapy applications. The TCR-like expression of this vector should achieve similar results compared to TCR—CAR gene replacement by genome edition but using a technology that has already been approved in clinic.

In summary, the technology described here, although less fine-tuned that genome edition tools, could render similar therapeutic benefits when applied to the patients and can be much easier to translate into the clinic.

Sequence Listing

SEQ ID No 1: AWE promoter containing a 387 bp
fragment of the WAS alternative promoter
immediately ″upstream″ of the 500 bp WAS proximal
promoter present in the WE vector.
Taagtcaaaggaggagagggcaacgcggtgggcaggagagaggccaacg
gccgcccggggcgaggggagccggtaggacgggaccaggactggccgac
ccggccccgcgcggggaagggggcgccttcctcccacaacacaaaacgg
tgcgcccgggttggccgcccctccccagtggtgcggccccgggtggacg
cttccgtgcgcgcgtccatgcccagccattgcgggctgcgggctccaag
ggtcgcacacgctggagagtgcaggttgccgggtccacccacagggctg
tagacacccctagggtcacacagacaaggctctggacacccacaggggc
acacacattggggagtgggcactcctgggctcacaaagactgagaatca
ctagtgaattcgggattacaggtgtgagctattgtccccagccaaaagg
aaaagttttactgtagtaacccttccggactagggacctcgggcctcag
cctcaggctacctaggtgctttagaaaggaggccacccaggcccatgac
tactccttgccacagggagccctgcacacagatgtgctaagctctcgct
gccagccagagggaggaggtctgagccagtcagaaggagatgggcccca
gagagtaagaaagggggaggaggacccaagctgatccaaaaggtgggtc
taagcagtcaagtggaggagggttccaatctgatggcggagggcccaag
ctcagcctaacgaggaggccaggcccaccaaggggcccctggaggactt
gtttcccttgtcccttgtggttttttgcatttcctgttcccttgctgct
cattgcggaagttcctcttcttaccctgcacccagagcctcgccagaga
agacaagggcagaaag
SEQ ID NO 2. 500 bp fragment of the proximal WAS
promoter
aattcgggattacaggtgtgagctattgtccccagccaaaaggaaaagt
tttactgtagtaacccttccggactagggacctcgggcctcagcctcag
gctacctaggtgctttagaaaggaggccacccaggcccatgactactcc
ttgccacagggagccctgcacacagatgtgctaagctctcgctgccagc
cagagggaggaggtctgagccagtcagaaggagatgggccccagagagt
aagaaagggggaggaggacccaagctgatccaaaaggtgggtctaagca
gtcaagtggaggagggttccaatctgatggcggagggcccaagctcagc
ctaacgaggaggccaggcccaccaaggggcccctggaggacttgtttcc
cttgtcccttgtggttttttgcatttcctgttcccttgctgctcattgc
ggaagttcctcttcttaccctgcacccagagcctcgccagagaagacaa
gggcagaaag
SEQ ID NO 3: 387 bp fragment of the WAS
alternative promoter
taagtcaaaggaggagagggcaacgcggtgggcaggagagaggccaacg
gccgcccggggcgaggggagccggtaggacgggaccaggactggccgac
ccggccccgcgcggggaagggggcgccttcctcccacaacacaaaacgg
tgcgcccgggttggccgcccctccccagtggtgcggccccgggtggacg
cttccgtgcgcgcgtccatgcccagccattgcgggctgcgggctccaag
ggtcgcacacgctggagagtgcaggttgccgggtccacccacagggctg
tagacacccctagggtcacacagacaaggctctggacacccacaggggc
acacacattggggagtgggcactcctgggctcacaaagactgagaatca
ctagtg

1. A polynucleotide comprising i) a nucleotide sequence encoding a specific chimeric antigen receptor (CAR) and ii) a promoter from the Wiskott-Aldrich syndrome locus or a fragment of said promoter comprising SEQ ID NO 2 or a nucleotide sequence having at least 70% identity with SEQ ID NO 2, wherein said promoter is operably linked to the nucleotide sequence encoding the CAR in order to drive the expression of the chimeric antigen receptor, and wherein the CAR comprises at least one extracellular ligand binding domain, a transmembrane domain and at least one intracellular signalling domain.

2. The polynucleotide of claim 1, wherein the promoter comprises SEQ ID NO 1 or a nucleotide sequence having at least 70% identity with SEQ ID NO 1.

3. The polynucleotide of claim 1, wherein the promoter is SEQ ID NO 1.

4. An expression vector comprising the nucleic acid of any of claims 1 to 3.

5. The expression vector of claim 4, wherein said expression vector is a viral vector.

6. The viral vector of claim 5, wherein said viral vector is a lentiviral vector.

7. Immune cells expressing at the cell surface membrane a specific chimeric antigen receptor comprising at least one extracellular ligand binding domain and at least one intracellular signalling domain wherein said Immune cells are transduced with the viral expression vector of any of claims 5 to 6.

8. Immune cells expressing at the cell surface membrane a specific chimeric antigen receptor comprising at least one extracellular ligand binding domain and at least one intracellular signalling domain wherein said chimeric antigen receptor is expressed by the expression vector of claim 4.

9. The immune cells according to any one of claims 7 to 8 derived from inflammatory T-lymphocytes, cytotoxic T-lymphocytes, regulatory T-lymphocytes or helper T-lymphocytes.

10. The immune cells according to any one of claims 7 to 9, wherein the cells are recovered from donors.

11. The immune cells according to any one of claims 7 to 8, wherein the cells are recovered from patients.

12. The immune cells according to any one of claims 7 to 11 for use in therapy.

13. The immune cells according to any one of claims 7 to 11 for use in the treatment of cancer, such as neoplasias, B-cell neoplasias, lymphoma or leukaemia, or multiple myeloma.
